(19) United States
(12) Patent Application Publication
(10) Pub. No.: US 2022/0015938 A1
(43) Pub. Date: Jan. 20, 2022

(54) Joint Orthosis with Helical Supporting Structure

(71) Applicants: Gregg Soifer, Dothan, AL (US); Sherayar Orakzai, Duluth, GA (US)

(72) Inventors: Gregg Soifer, Dothan, AL (US); Sherayar Orakzai, Duluth, GA (US)

(21) Appl. No.: 16/930,024

(22) Filed: Jul. 15, 2020

(51) Int. Cl. A61F 5/01

(52) U.S. Cl. CPC A61F 5/0123; A61F 2005/0179; A61F 2005/0172

(57) ABSTRACT

A helical orthosis is an apparatus to support the function of an injured or impaired joint with a helical supporting structure mounted about the affected joint. The apparatus includes at least one helical coil, a primary band, and a secondary band in the preferred embodiment. A first end of the helical coil is slidably mounted to the primary band with the helical coil mounted in between the primary band and the secondary band along a helical path, wherein he helical coil exerts force against the primary band and the secondary band is support of the intermediate affected joint. Accordingly, a second end of the helical coil is slidably mounted to the secondary band contralateral to the first end of the helical coil. This configuration will enable a user to adjust the force exerted on the affected joint by traversing the helical coil about the limb into any desirable configuration.

### FIELD OF THE INVENTION

The present invention relates generally to the field of medical braces and similar supporting devices. More specifically, the present invention provides a wearable brace suitable for supporting the normal function and range of motion of a user's limb without exerting torsion across an affected joint.

### BACKGROUND OF THE INVENTION

Conventional knee braces employed in conjunction with physical therapy (or the like) provide a fixed axis-of-rotation to mimic bending at the knee joint. However, bending at the knee joint is accomplished via a rolling motion which, at each instant during the motion, is associated with an instantaneous axis-of-rotation. From the perspective of the femur (thighbone), the tibial plateau (top of shinbone) rolls on the femoral condyles (medial and lateral rounded ends of femur). This type of mechanical arrangement may be mimicked by a simplified pivoting hinge but cannot be perfectly replicated by such an assembly. Accordingly, the fixed axis-of-rotation of the conventional brace is, in general, not align with the actual instantaneous axis-of-rotation of the knee joint creating torsion in the knee joint during bending motions. This torsion is transmitted to neighboring joints such as the hip joint causing both pain and structural damage, particularly in long-term applications wherein a user with a compromised joint may rely on such a conventional brace to maintain personal mobility. This torsion is further amplified if the brace becomes misaligned during use, either by the abnormal motion of the simple hinge relative to the user's own biology, or by the user misadjusting the conventional brace due to any discomfort such a mismatch may cause.

The present invention aims to provide a torsion-free support to a user's joints by the insertion of a helical interstitial structure spanning across the affected joint. According to the preferred embodiment the present invention is suitable for fixation about a user's thigh and shin, though it is generally considered that a similar, rescaled iteration of the apparatus described herein may be applied to any other areas of the body without limitation. The specific dimensions and manufacture of the present invention are variable across multiple iterations and use cases such that the resistive force provided by the present invention across any given degree of movement may be affected by irregular construction and internal static forces emergent from said irregular construction. More specifically, the present invention may be deformed at various positions to alter the resistive or assistive forces applied across a joint.

### BRIEF DESCRIPTION OF THE DRAWINGS

FIG. 1 is an isometric view of the present invention from the top-front-left perspective.

FIG. 2 is an isometric view of the present invention from the bottom-rear-right perspective.

FIG. 3 is front elevational view of the present invention.

FIG. 4 is a rear elevational view of the present invention, wherein a sectional view of an exemplary helical coil is taken along line A-A.

FIG. 5 is a right-side elevational view of the present invention.

FIG. 6 is a plan view of the present invention from the top perspective.

### DETAILED DESCRIPTION OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

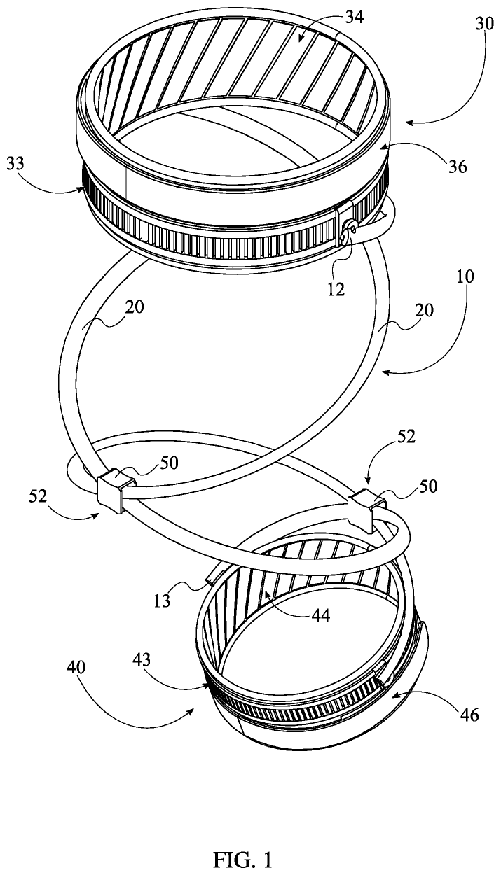

As can be seen in FIG. 1 through 6, the present invention is a helical orthosis utilized to support an injured or weakened joint by providing an external helical load-bearing structure and supporting attachments to a user's body. The specific adjustable construction of the load-bearing structure and the supporting attachments in considered to produce a superior form of brace to those currently available or presently known in the field. Accordingly, though the present invention is specifically aimed towards use is relation to knees, a similar iteration of the present invention may be employed in support of any area of a body without departing from the original spirit and scope of the present invention.

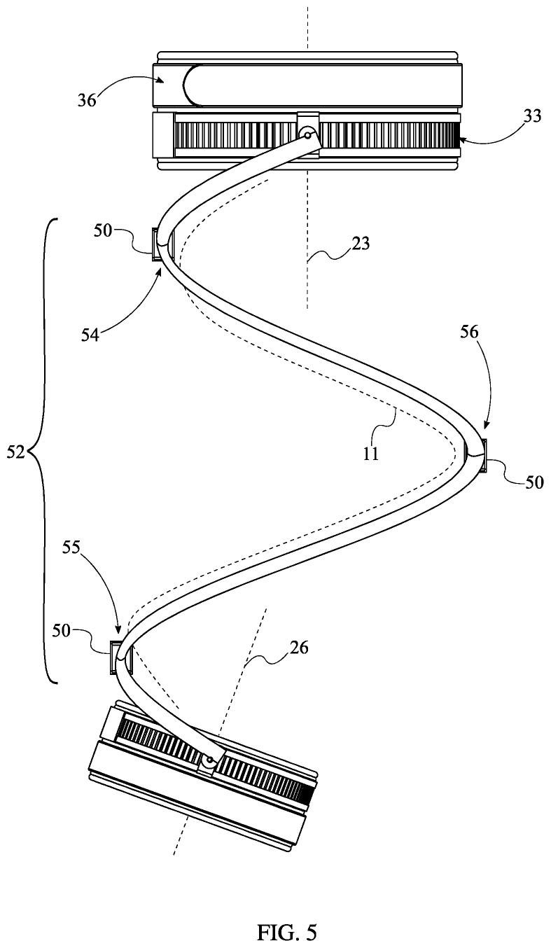

In a preferred embodiment, the helical orthosis comprises at least one helical coil 10, a primary band 30, and a secondary band 40. The helical coil 10 defines the load-bearing structure previously mentioned, arranged such that the helical coil 10 is mounted between the primary band 30 and the secondary band 40 along a helical path 11. As shown in FIG. 5, the helical path 11 defines an unobstructed space between the primary band 30 and the secondary band 40 with suitable clearance such that the helical coil 10 may deflect, compress, or otherwise deform to absorb forces otherwise exerted across a user's knee without directly contacting the supported joint. The primary band 30 and secondary band 40 define mutually similar components serving as attachments to a user's body, ideally the thigh and shin, respectively. In this arrangement the helical coil 10 spans between the primary band 30 and the secondary band 40 to enclose the user's knee without obstructing or impeding the full range of normal motion of the joint. It is further considered that the position of the helical coil 10 about the user's knee may be adjusted to ensure proper fit and function, including the clearance for normal motion as outlined. According to this functionality, a first end of the helical coil 12 is slidably mounted to the primary band 30. Likewise, a second end of the helical coil 13 is slidably mounted to the secondary band 40. When operated in conjunction, these adjustable fixtures enable a user to configure the present invention to fit across any joint provided a suitably scaled instance of the helical coil 10.

In another potential embodiment considered within the scope of the present invention, the present invention further comprises a plurality of primary mounts 31 and a plurality of secondary mounts 41. Further, in this embodiment, the at least one helical coil 10 comprises a plurality of helical coils 20. The plurality of primary mounts 31 ideally defines a series of socket couplings situated between the plurality of helical coils 20 and the primary band 30, the plurality of secondary mounts 41 defining a similar structure positioned between the plurality of helical coils 20 and the secondary band 40. The arrangement of these flexible connections allows the plurality of helical coils 20 to deflect and pivot in relation to the primary band 30 and the secondary band 40 without applying torsion across the corresponding primary mount 32 or the corresponding secondary mount 42. Consequently, this arrangement enables minute movements of the patient's limb while maintaining positional support of a joint via the plurality of helical coils 20. According to this preferred embodiment, the first end of each the plurality of helical coils 20 is laterally mounted onto the primary band 30 by a corresponding primary mount 32 from the plurality of primary mounts 31. Similarly, the second end of each the plurality of helical coils 20 being laterally mounted onto the secondary band 40 by a corresponding secondary mount 42 from the plurality of secondary mounts 41. The lateral mounting of the plurality of helical coils 20 to the primary band 30 and secondary band 40 as described creates a roughly cylindrical profile to the assembled embodiment of the present invention, wherein the plurality of helical coils 20 does not impede the regular positioning of a user's limb within the roughly cylindrical profile between the primary band 30 and the secondary band 40.

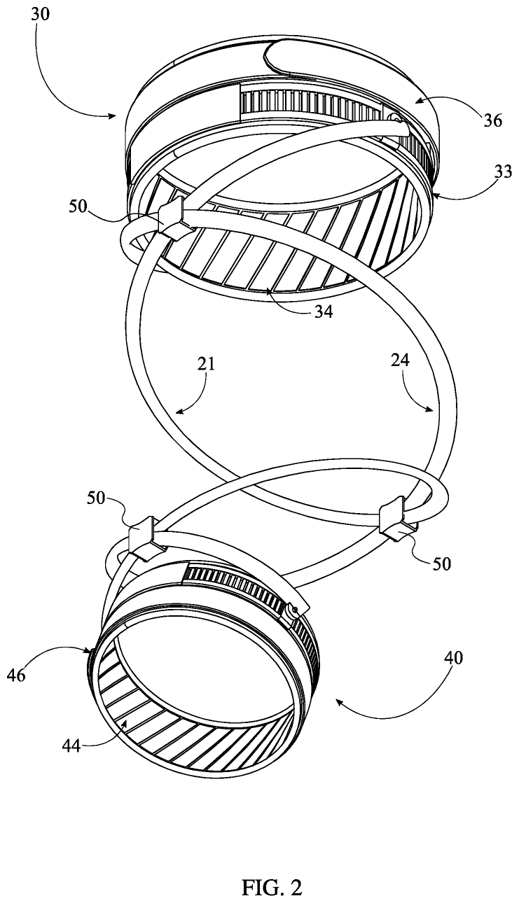

It is further proposed that opposing forces in tension provide a suitable support structure for an injured joint that is naturally resistant to disruptive torsional forces created when a patient moves said joint. In the preferred embodiment of the present invention illustrated in FIGS. 2 and 5, a helical axis of a first coil 22 is oriented opposite to a helical axis of a second coil 25, wherein the first coil 21 and the second coil 24 are from the plurality of helical coils 20. In general terms, helical axes ordinarily define the axis of rotation and the line along which translation of a body occurs in three dimensions. Thus, the present invention comprises an entwined counter-wound cylindrical structure in which a user's limb is supported by the composite spring force of the plurality of coils 20. In this embodiment, the first coil 21 and the second coil 24 are configured to cross each other at multiple positions along the normal path of each helical coil 10. Supporting this configuration, the first end of the first coil 21 is positioned offset from the first end of the second coil 24 around the primary band 30 and the second end of the first coil 21 is positioned offset from the second end of the second coil 24 around the secondary band 40. The radial offset of the terminal ends of the first coil 21 and the second coil 24 are arranged in this way to ensure that the locations of the intersections of the plurality of coils 20 do not interfere with the normal range of motion of the supported limb.

To further minimize discomfort and disruption of the normal function of the joint, it is proposed that at least one coil guide 50 is slidably mounted onto an intersection point 51 between the first coil 21 and the second coil 24. The coil guide 50 ideally defines an enclosed fitting of suitable size and material quality to capture both the first coil 21 and the second coil 24 simultaneously, physically establishing the intersection point 51 between both separately mobile structures.

In a related configuration of the present invention, at least one coil guide 50 is proposed to comprise a plurality of coil guides 50. This configuration additionally defines a helical axis of at least one first coil 21 crossing a helical axis of at least one second coil 24 at a plurality of intersections 52, wherein the first coil 21 and the second coil 24 are from the plurality of helical coils 20. Each of the plurality of coil guides 50 is slidably mounted onto the first coil 21 and the second coil 24 at a corresponding intersection 53 from the plurality of intersections 52 in a similar arrangement to the configuration outlined previously. The plurality of intersections 52 is ideally positioned outside of the normal range of motion for a supported limb, thereby enabling the present invention to define a consistent structural profile without impeding the normal function of a limb. In the proposed configuration a knee may be supported by a primary intersection 54 positioned proximal to the primary band 30, wherein the primary intersection 54 is from the plurality of intersections 52. This primary intersection 54 is positioned roughly behind a user's thigh in an exemplary embodiment, above the knee and outside of the space occupied by said user's calf during maximum positive flexion (i.e. bending the knee). Additionally, a secondary intersection 55 is positioned proximal to the secondary band 40, wherein the secondary intersection 55 is from the plurality of intersections 52. The secondary intersection 55 is positioned behind the user's calf in the exemplary application, also outside of the space occupied by a user's thigh during maximum positive flexion. Finally, at least one intermediary intersection 56 is positioned in between the primary intersection 54 and the secondary intersection 55, wherein the intermediary intersection 56 is from the plurality of intersections 52. The exemplary application features the intermediary intersection 56 adjacent to the patella or kneecap, also clear of any normal range of motion for a human leg. Supporting this arrangement, the coil guide 50 is slidably mounted onto an intersection point 51 between a first coil 21 and a second coil 24, wherein the first coil 21 and the second coil 24 are from the plurality of helical coils 20. In the exemplary application outlined thus far an instance of the coil guide 50 will be positioned at each of the plurality of intersections 52 to prevent the first coil 21 and the second coil 24 from rattling against one another or becoming separated during movement of the limb.

It is considered that the present invention may require reconfiguration to fit a variety of patients, both in terms of limb dimensions and desired supporting force exerted by the plurality of helical coils 20. Supporting this adjustable functionality, the present invention further comprises a first radial adjuster 33 and a second radial adjuster 43 as illustrated in FIG. 1. Both components define similar releasable fixtures enabling the translation of at least one helical coil 10 about the primary band 30 and the secondary band 40. In the preferred embodiment the first radial adjuster 33 is integrated in between the first end of the helical coil 12 and the primary band 30 and the second radial adjuster 43 is integrated in between the second end of the helical coil 13 and the secondary band 40. This arrangement will enable a user to adjust the preload of the helical coil 10 by winding or unwinding the helical coil 10 between the primary band 30 and the secondary band 40, thereby increasing or decreasing the force exerted across a user's joint by said helical coil 10. This reconfiguration may also be prudent when fitting the present invention to a new patient after adjusting the diameter of the primary band 30 or the secondary band 40, presuming that the position of the helical coil 10 will be affected by such an adjustment.

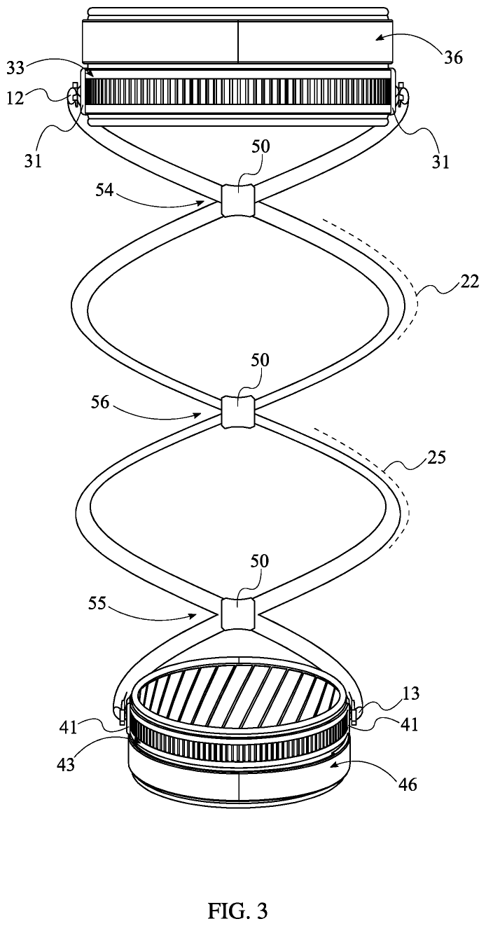

As outlined above, the primary band 30 and the secondary band 40 are adjustable to comfortably fit any patient. As illustrated in FIG. 3, this functionality is achieved by integrating a primary adjustment mechanism 36 along the primary band 30 to modify the circumference of the primary band 30. The primary adjustment mechanism 36 ideally comprises a screw-adjustable band clamp mounted around a user's leg, though it is also contemplated that any means of adjusting the effective inner diameter of the primary band 30 may constitute the primary adjustment mechanism 36 across multiple embodiments. In a related embodiment, a secondary adjustment mechanism 46 is also integrated along the secondary band 40 to modify the circumference of the secondary band 40. The use and configuration of the secondary adjustment mechanism 46 is generally similar to the primary adjustment mechanism 36 outlined previously. The adjustment of the relative circumferences of the primary band 30 and the secondary band 40 will ideally result in the helical coil 10 tapering from the primary band 30 toward the secondary band 40 with a maximal circumference of the primary band 30 being greater than a maximal circumference of the secondary band 40. This configuration is proposed to match the anatomical dimensions of any given limb more accurately, i.e. a limb generally tapers away from the torso from a first segment (thigh or upper arm) across a joint (knee or elbow) to a second segment (calf or forearm).

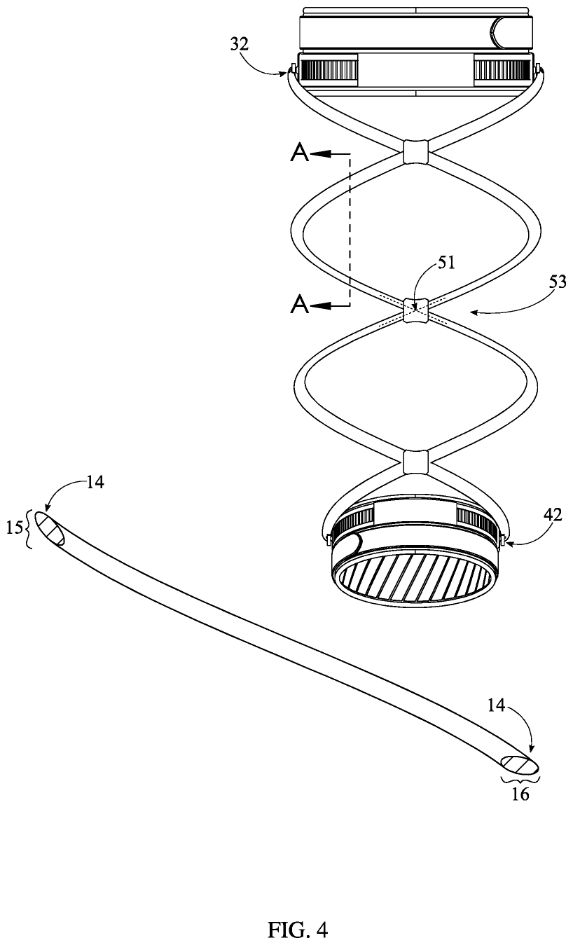

The helical structure of at least one helical coil 10 supported between the primary band 30 and the secondary band 40 inherently provides a measure of support to a joint positioned within said helical coil 10. As illustrated in FIG. 4, the helical coil 10 comprises a lateral cross-sectional profile 14, defining the planar dimensions of a single section of the helical coil 10. This lateral cross-sectional profile 14 is hyperelliptical in at least one embodiment, wherein a transverse dimension 15 of the lateral cross-sectional profile 14 is greater than an orthogonal transverse dimension 16 of the lateral cross-sectional profile 14. This structural irregularity provides targeted deformation resistance to the spring-like action of the helical coil 10 by creating a material elongation along an axis of targeted resistance. In practice, the transverse dimension 15 may define a material elongation parallel to a user's limb to prevent compression of the helical coil 10 at the site of said elongation. Conversely, the transverse dimension 15 may be defined perpendicular to a user's limb to allow linear compression of the helical coil 10 between the primary band 30 and the secondary band 40 to be pronounced along the affected length of the helical coil 10 while lateral deflection is minimized.

In addition to the variable contours described above, structural deformations defined along the entire length of at least one helical coil 10 are proposed to mimic the natural resting position of a limb. In an application supporting the knee of a patient the helical coil 10 forms an arch between the primary band 30 and the secondary band 40 to approximate the profile of a relaxed leg. In broader terms the present invention comprises a primary axis 23 and a secondary axis 26, the secondary axis 26 being angularly offset from the primary axis 23. Presuming an alignment of the primary band 30 and the secondary band 40 with adjacent portions of a limb separated by a joint, the primary band 30 is concentric to the primary axis 23 and the secondary band 40 is concentric to the secondary axis 26. This arrangement will position the intersection of the primary axis 23 and the secondary axis 26, the approximate center of rotation of the joint, and the apex of the arched helical coil 10 at approximately the same position. Consequently, the helical coil 10 will float freely around the limb during the full range of motion of said limb while biasing the joint into a relaxed posture intermediate between a maximum flexion and extension.

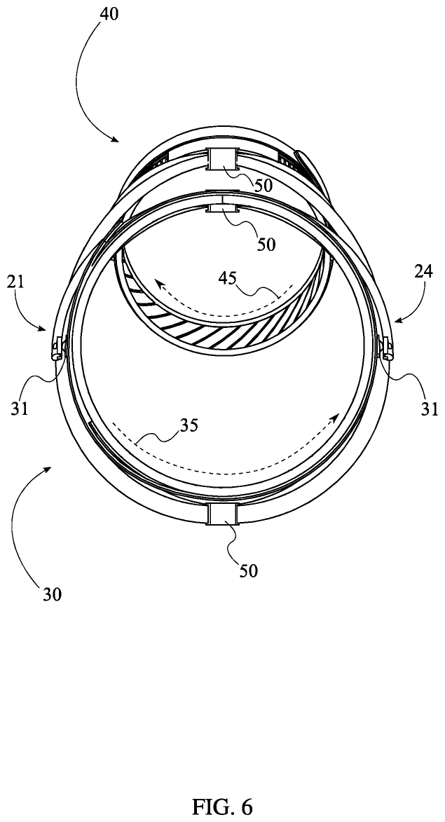

Incidental to the linear forces exerted across the primary band 30 and the secondary band 40 by the compression of the helical coil 10, it is also understood that a torsional force is exerted by iterations utilizing the singular helical coil 10. This torsional force may unintentionally ratchet the primary band 30 or secondary band 40 into an uncomfortable position on a user's limb, in addition to disrupting any configurations related to the support provided to a joint by the compression of the helical coil 10. To counteract this torsional force, the present invention comprises a first volume of padding 34 and a second volume of padding 44, wherein the first volume of padding 34 is laterally connected within the primary band 30 and the second volume of padding 44 being laterally connected within the secondary band 40. This arrangement permits the tightening of the primary band 30 and the secondary band 40 around a patient's limb to a greater extent than bare iterations of the same components without causing undue discomfort to a patient. Further, the first volume of padding 34 is dispersed about the interior of the primary band 30 with a primary directional bias 35 and the second volume of padding 44 is dispersed about the interior of the secondary band 40 with a secondary directional bias 45, wherein the secondary directional bias 45 is opposed to the primary directional bias 35. As shown in FIG. 6, the counter-arrangement of the first volume of padding 34 and the second volume of padding 44 ensures that any torsional forces exerted by the compression of the helical coil 10 are directly counteracted by the intentional collapse of the first volume of padding 34 or the second volume of padding 44, whichever is arranged in opposition to the direction of the torsional force. An exemplary embodiment of the first volume of padding 34 or the second volume of padding 44 defines a series of faceted pads that pivot against the corresponding band in response to rotation about a patient's limb. If the direction of rotation is against the corresponding directional bias of the corresponding volume of padding temporarily reduces the inner diameter of the corresponding band, thereby constricting the patient's limb and preventing the displacement of the corresponding band. It is additionally considered that the primary directional bias 35 and the secondary directional bias 45 may constitute a material flexibility across a single radial vector, achieving the same temporary ratcheting effect.

Although the invention has been explained in relation to its preferred embodiment, it is to be understood that many other possible modifications and variations can be made without departing from the spirit and scope of the invention as hereinafter claimed.

What is claimed is:

1. A helical orthosis comprising:
at least one helical coil;
a primary band;
a secondary band;
a first end of the helical coil being slidably mounted to the primary band;
the helical coil being mounted in between the primary band and the secondary band along a helical path; and
a second end of the helical coil being slidably mounted to the secondary band, contralateral to the first end of the helical coil.

2. A helical orthosis as claimed in claim 1 comprising:
a plurality of primary mounts;
a plurality of secondary mounts;
the at least one helical coil being a plurality of helical coils;
the first end of each the plurality of helical coils being laterally mounted onto the primary band by a corresponding primary mount from the plurality of primary mounts; and
the second end of each the plurality of helical coils being laterally mounted onto the secondary band by a corresponding secondary mount from the plurality of secondary mounts.

3. A helical orthosis as claimed in claim 2 comprising:
a helical axis of a first coil being oriented opposite to a helical axis of a second coil, wherein the first coil and the second coil are from the plurality of helical coils;
the first end of the first coil being positioned offset from the first end of the second coil around the primary band; and
the second end of the first coil being positioned offset from the second end of the second coil around the secondary band.

4. A helical orthosis as claimed in claim 3 comprising:
at least one coil guide; and
the coil guide being slidably mounted onto an intersection point between the first coil and the second coil.

5. A helical orthosis as claimed in claim 2 comprising:
a plurality of coil guides;
a helical axis of the at least one first coil crossing a helical axis of at least one second coil at a plurality of intersections, wherein the first coil and the second coil are from the plurality of helical coils;
each of the plurality of coil guides being slidably mounted onto the first coil and the second coil at a corresponding intersection from the plurality of intersections;
a primary intersection being positioned proximal to the primary band, wherein the primary intersection is from the plurality of intersections;
a secondary intersection being positioned proximal to the secondary band, wherein the secondary intersection is from the plurality of intersections;
at least one intermediary intersection being positioned in between the primary intersection and the secondary intersection, wherein the intermediary intersection is from the plurality of intersections; and
the coil guide being slidably mounted onto an intersection point between a first coil and a second coil, wherein the first coil and the second coil are from the plurality of helical coils.

6. A helical orthosis as claimed in claim 1 comprising:
a first radial adjuster;
a second radial adjuster;
the first radial adjuster being integrated in between the first end of the helical coil and the primary band; and
the second radial adjuster being integrated in between the second end of the helical coil and the secondary band.

7. A helical orthosis as claimed in claim 1 comprising:
a primary adjustment mechanism; and
the primary adjustment mechanism being integrated along the primary band, wherein the primary adjustment mechanism modifies a circumference of the primary band.

8. A helical orthosis as claimed in claim 1 comprising:
a secondary adjustment mechanism; and
the secondary adjustment mechanism being integrated along the secondary band, wherein the secondary adjustment mechanism modifies a circumference of the secondary band.

9. A helical orthosis as claimed in claim 1 comprising:
the helical coil tapering from the primary band toward the secondary band; and
a maximal circumference of the primary band being greater than a maximal circumference of the secondary band.

10. A helical orthosis as claimed in claim 1 comprising:
the helical coil comprising a lateral cross-sectional profile; and
the lateral cross-sectional profile being hyperelliptical, wherein a transverse dimension of the lateral cross-sectional profile is greater than an orthogonal transverse dimension of the lateral cross-sectional profile.

11. A helical orthosis as claimed in claim 1 comprising:
a primary axis;
a secondary axis,
the secondary axis being angularly offset from the primary axis;
the primary band being concentric to the primary axis; and
the secondary band being concentric to the secondary axis.

12. A helical orthosis as claimed in claim 1 comprising:
a first volume of padding;
a second volume of padding;
the first volume of padding being laterally connected within the primary band;
the second volume of padding being laterally connected within the secondary band;
the first volume of padding being dispersed about the interior of the primary band with a primary directional bias; and
the second volume of padding being dispersed about the interior of the secondary band with a secondary directional bias, wherein the secondary directional bias is opposed to the primary directional bias.
